(19) United States
(12) Patent Application Publication
(10) Pub. No.: US 2020/0118658 A1
(43) Pub. Date: Apr. 16, 2020

(54) SYSTEM AND METHOD FOR TRANSFERRING AND POPULATING PHARMACY DATA INTO A MOBILE APPLICATION

(71) Applicants: Melanie N. Kalashian, Las Vegas, NV (US); Michael N. Kalashian, Las Vegas, NV (US)

(72) Inventors: Melanie N. Kalashian, Las Vegas, NV (US); Michael N. Kalashian, Las Vegas, NV (US)

(21) Appl. No.: 16/591,479

(22) Filed: Oct. 2, 2019

Related U.S. Application Data
(60) Provisional application No. 62/744,468, filed on Oct. 11, 2018.

(51) Int. Cl. G16H 10/65; G16H 20/10; G06F 9/451; H04W 12/06; H04W 12/08; H04W 4/20

(52) U.S. Cl. CPC G16H 10/65; G16H 20/10; G06F 9/451; G06Q 30/0207; H04W 12/08; H04W 4/20; H04W 12/06

(57) ABSTRACT

A prescription mobile application downloadable on mobile smart devices. The mobile application enrolls one or more pharmacies into one or more data exchange programs. The data exchange installs a custom designed data extraction harmonization program on any enrolled pharmacy database. When a user contacts a pharmacy for his or her prescription drugs, the data extraction harmonization program creates the user's harmonized data file and, upon user request through the application, sends the same to the user's mobile application. The data population of the user's mobile device requires the user to be a customer of the pharmacy but does not require the user to register a user account with a pharmacy's web or mobile interface. The mobile application can integrate the data from the pharmacies into one prescription reminder calendar, set alarms, record usage, warn for reordering and display warnings, pharmacy discounts/sales, new recipes and any other custom data.

### CROSS REFERENCE

This application claims priority to U.S. Provisional Patent Application No. 62/744,468 filed Oct. 11, 2018 which is incorporated herein by reference for all purposes.

### FIELD OF THE INVENTION

The embodiments of the present invention relate to the automatic downloading of pharmacy prescription data into a harmonized format accessible on a mobile application (aka “APP”).

### BACKGROUND

Several previous mobile applications provide prescription reminders, usage alerts and reorder alarms to a user. The systems generally require the user to manually input each prescription, dosage, side effects, warnings about what not to mix with other doses such as a blood thinner or take with a meal, etc., the pharmacy, frequency (e.g., two times a day), alternate pharmacies and so forth. Many users, especially the elderly who may need a dozen prescriptions a day, cannot easily enter all this data properly on their smart phone or tablet, and are prone to making mistakes. Keeping up with changing prescriptions can also be troublesome.

A currently-available medicine tracking mobile application known as Medisafe Project Ltd. or MediSafe, offers users the option of importing pharmacy data into a mobile application.

The MediSafe application attempts to spoof the registration form of the subject pharmacy (e.g., CVS, Walgreens, RiteAid, etc.) that provide user prescription data. For example, the user must first register (i.e., sign-up) with the pharmacy's platform and create a username and password. Then, the user needs to sign-in to his or her pharmacy account through the MediSafe mobile application in order to extract their prescription information. With this method, the user may not have received approval from the pharmacy to interact with the user's pharmacy account dashboard because the MediSafe application accesses the pharmacy platform using the user's pre-registered pharmacy account login. Once the application has gained access to the user's pre-registered pharmacy account, the application then scrapes information from the pharmacy user account interface and transmits to the user's mobile application interface. The data queried into the application is restricted to the standard output report available from the pharmacy's website account platform.

What is needed is a more robust interface with secure pharmacy alliance to exchange the maximum amount of data, including images, from any pharmacy to any mobile device. In addition, the interface, system, method should be capable of transferring data from the pharmacy requiring a user to pre-register with the pharmacy's website interface, mobile application(s), and/or online systems.

The embodiments of the present invention include each pharmacy (e.g., CVS, Walgreens, Walmart, etc.) pre-approving information that is selected from their database to be sent to the mobile application. The pharmacy normalizes the data to the format that is provided to them from the mobile application according to the embodiments of the present invention. When the pharmacy receives the security details, the pharmacy checks its database records and returns data in a predefined file format that has been harmonized between the pharmacy and the mobile application.

### SUMMARY

Prescription information is highly personal medical information and is protected by Federal HIPPA laws. A patient or user owning a smart phone or mobile device and having access to the Internet will appreciate acquiring copies of prescription and related information from their pharmacy. There are many ways to do this, for example, call their pharmacy or examine their prescription bottles or pharmacy printouts. All of which require entering information into an application on their mobile device or smart phone via the user interface. Such a method is prone to errors and may not include all medicines and/or information regarding each medicine may not be complete. At a minimum, such a method is risky and at worst dangerous especially for the elderly and other vulnerable persons in our population.

The embodiments of the present invention provide a method to exchange a user's personal medical records (i.e., prescription records) in a secure, reliable and efficient manner. A primary aspect of the embodiments of the present invention is to recruit a pharmacy or plurality of pharmacies such as CVS, Walgreens, Walmart, etc. to incorporate secure access from the mobile application of the embodiments of the present invention to their pharmacy database.

Another aspect of the embodiments of the present invention is to provide at least a dual security check between the mobile application and the pharmacy, including: (i) confirming the user exists in the pharmacy database meaning confirming the user is a customer and has prescription record(s) with the pharmacy and (ii) verifying the identity of the user inquiring.

Another aspect of the embodiments of the present invention is to allow a user to use multiple mobile devices (e.g., smart phone and tablet).

Another aspect of the embodiments of the present invention is to allow access to a pharmacy or plurality of pharmacy databases with no logins to any pharmacy system as a requirement to access user's pharmacy records.

Another aspect of the present invention is the option to include providing custom data such as but not limited to, a picture of a pharmacist, marketing and sales promotional materials, discounts, new recipes or recommended food to eat with a particular medicine, from the pharmacy into the mobile application.

Another aspect of the embodiments of the present invention is to process various prescriptions from different pharmacies into a single coordinated daily reminder alarm system.

Another aspect of the embodiments of the present invention is to harmonize all incoming raw data into a single user format and provide a uniform history reporting system which may encompass a variety of pharmacies.

Other aspects of the embodiments of the present invention appear from the following description and appended claims, reference being made to the accompanying drawings forming a part of this specification wherein like reference characters designate corresponding parts in the several views.

### BRIEF DESCRIPTION OF THE DRAWINGS

FIG. 1 illustrates a block diagram for obtaining pharmacy data for an individual according to the embodiments of the present invention;

FIG. 2 illustrates a flow diagram of the steps for downloading patient user data according to the embodiments of the present invention;

FIG. 3 illustrates a block diagram of a previous solution (prior art) to obtain individual data from a pharmacy through a webpage;

FIG. 4 illustrates a chart detailing data fields according to the embodiments of the present invention;

FIGS. 5A and 5B illustrate a pair of charts detailing the table definitions of the data structures according to the embodiments or the present invention;

FIGS. 6A through 6D illustrate screen shots detailing actual flow of registering a new user, downloading data and setting a reminder alarm according to the embodiments of the present invention; and

FIG. 7 illustrates a chart detailing the flow of a new user connecting to the Internet and downloading the mobile application to connect with pharmacy data for import into the mobile application according to the embodiments of the present invention.

### DETAILED DESCRIPTION

For the purposes of promoting an understanding of the principles in accordance with the embodiments of the present invention, reference will now be made to the embodiments illustrated in the drawings and specific language will be used to describe the same. It will nevertheless be understood that no limitation of the scope of the invention is thereby intended. Any alterations and further modifications of the inventive feature illustrated herein, and any additional applications of the principles of the invention as illustrated herein, which would normally occur to one skilled in the relevant art and having possession of this disclosure, are to be considered within the scope of the invention claimed.

Those skilled in the art will recognize that the virtual, digital and online embodiments of the present invention involve both hardware and software elements which portions are described below in such detail required to construct and operate a game prescription import method and system according to the embodiments of the present invention.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.), or an embodiment combining software and hardware. Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer drive, a hard disk, a random access memory (RAM), a read-only memory (ROM), an erasable programmable read-only memory (EPROM or Flash memory), an optical fiber, a portable compact disc read-only memory (CD-ROM), and optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied thereon, for example, in baseband or as part of a carrier wave. Such a propagated signal may take any variety of forms, including, but not limited to, electromagnetic, optical, or any suitable combination thereof. A computer readable signal medium may be any computer readable medium that is not a computer readable storage medium and that can communicate, propagate, or transport a program for use by or in conjunction with an instruction execution system, apparatus, or device.

Program code embodied on a computer readable medium may be transmitted using any appropriate medium, including but not limited to wifi, wireless, wireline, optical fiber cable, RF and the like, or any suitable combination of the foregoing.

Computer program code for carrying out operations for aspects of the present invention may be written in any combination of one or more programming languages, including an object oriented programming language such as Java, Swift, Apple Script Smalltalk, C++ or the like or conventional procedural programming languages, such as the “C” programming language, AJAX, PHP, HTML, XHTML, Ruby, Objective C++, Python CSS or similar programming languages with a variety of programming models, including but not limited to Cocoa, Carbon, Java etc. The programming code may be configured in an application, an operating system, as part of a system firmware, or any suitable combination thereof. The programming code may execute entirely on the user's smart phone or mobile device, partly on the user's smart phone or mobile device, as a standalone software package, partly on the user's smart phone or mobile device and partly on a remote computer or entirely on a remote computer or server as in a client/server relationship sometimes known as cloud computing. In another scenario, the remote computer may be connected to the user's smart phone or mobile device, or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions or algorithms. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram.

The computer program instructions may also be loaded onto a computer, other programmable data processing apparatus, or other devices to cause a series of operational steps to be performed on the computer, other programmable apparatus or other devices to produce a computer-implemented process such that the instructions which execute on the computer or other programmable apparatus provide processes for implementing the functions/acts specified in the flowchart and/or block diagrams.

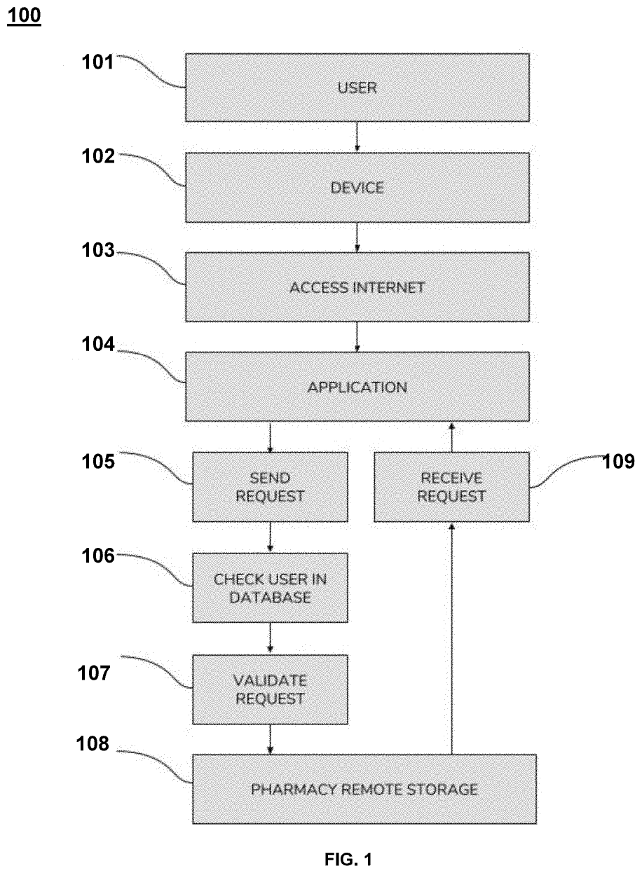

A block diagram 100 is shown in FIG. 1. As shown, the user 101 connects using a device 102 that may include but is not limited to an application running on a mobile device such as smart phone or tablet. Device 102 is configured to connect 103 to the Internet allowing the application 104 running on the mobile device to communicate with database servers 108 associated with participating pharmacies. The application 104 is configured to send a request 105 for data to one or more pharmacies via the database servers. A first security step involves the application validating against the database server by sending a security code and/or process to authorize access to the database server and confirm that the individual for whom data is being requested exists in a subject database 106. If the user record exists in database 106 and the identifying fields match, a second security step is initiated whereby the application requests the user to validate his or her identity via answering security questions, logging into to a pharmacy account, or other such methods that the pharmacy database server must validate 107. Only after both security protocols are satisfied does the request reach the subject database 106, which responds by sending subject data in a pre-authorized format 109 to the application 104 on the user's device.

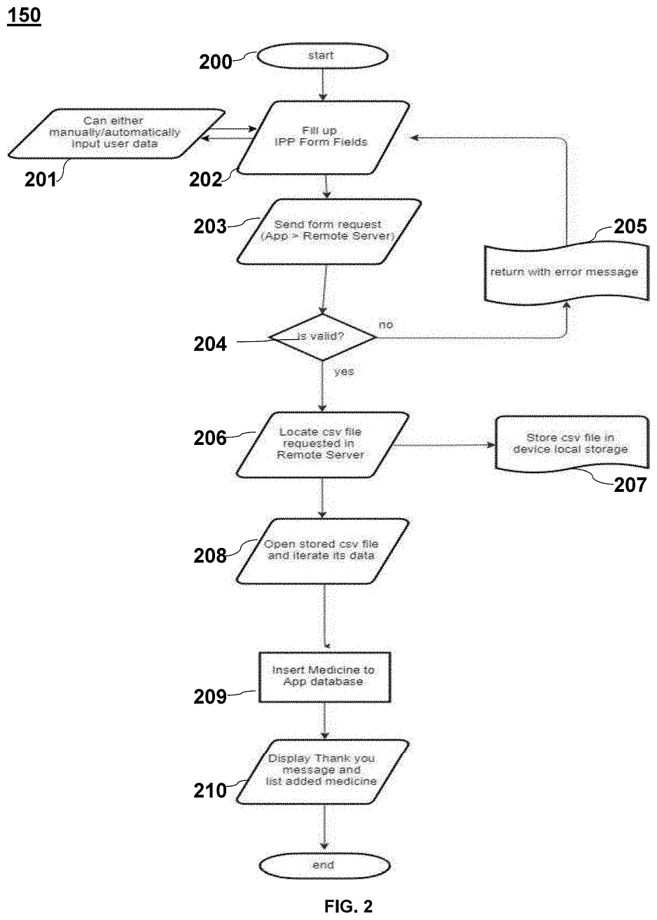

A step-by-step diagram 150 of the connection method is shown in FIG. 2. When the user opens 200 the application, the first option is to configure the user's prescription data in the application. This can be done manually or automatically 201 using the connection to the pharmacy databases. The method and connection to the pharmacy database is referred to herein as Import Pharmacy Prescriptions (IPP). If the user chooses to use IPP to download the data automatically, the user must first enter identifying information for the patient 202. The application also sends a specific code that identifies the application to the pharmacy database 203. This data request is sent to the pharmacy database 203, where the two-step authorization is performed 204. This two-step process first validates that the application is authorized to connect to the pharmacy database and verifies the patient exists in the pharmacy database and second verifies the authentication of the user identity from the pharmacy 204. If either of these security checks fails, an error message 205 is returned and the user is returned to the entry screen. If the security checks are satisfied, the patient data in the pharmacy database are downloaded 206 to the user's device running the application, where the data is stored locally 207. The application then opens the received data file 208 and reads/writes it into a data structure within an application database 209. At this time, the user has completely populated the local application database with his or her pharmacy data. A confirmation is displayed 210 and the user may edit and modify data, set reminders and/or alarms as desired.

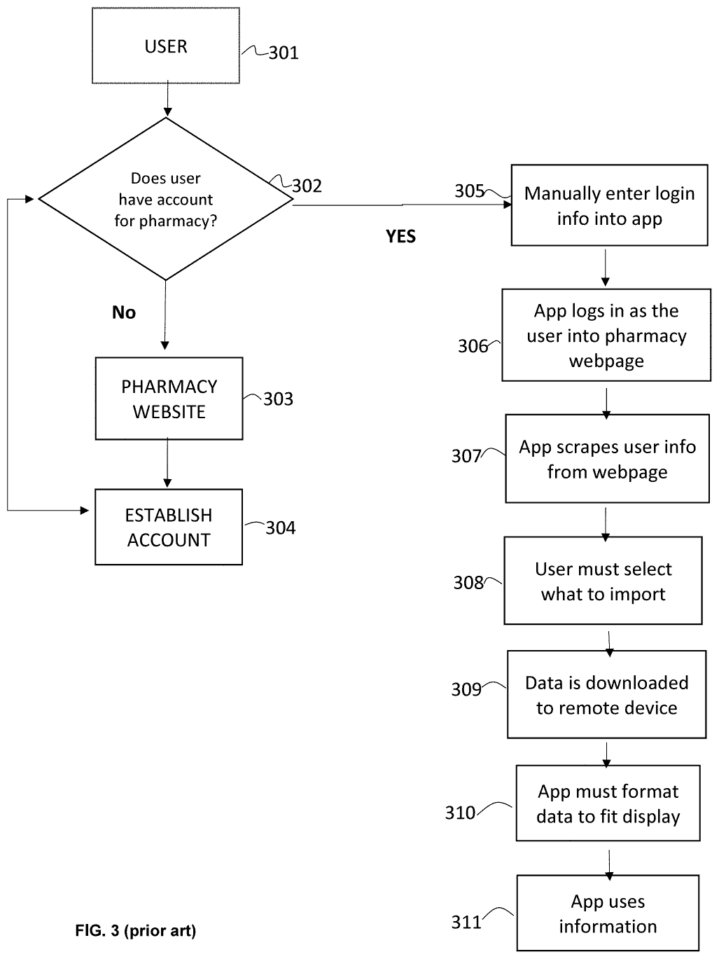

The operational steps of the previous systems (prior art) are shown in FIG. 3. Via a remote system or device, a user 301 opens an application to retrieve personal data from a pharmacy. The application then needs to confirm 302 the user's login and password associated with the pharmacy. If it does not confirm, the user must manually (i.e., outside of the application) go to the pharmacy's website 303 and establish an account 304. This may require several different steps specific to the pharmacy. Once the user has established the account, he or she must then return to the pharmacy application and proceed to enter the security information manually 305 into the application. When this is complete, the application logins to the pharmacy's webpage as the user 306 and attempts to navigate to a page showing the prescription data. At this page, the application then reads the data over the network connection and presents it to the user 307. This may not be possible if the pharmacy has changed the configuration of the webpage subsequent to the current version of the application that the user downloaded previously. If the application is successful, the user then selects which records to import 308 to start the download of the data 309. There is no guarantee that the data maintained by any individual pharmacy is in the same format or structure compatible with the remote application. Thus, a format check and possible change may be required 310. Only then is the data usable 311 by the user.

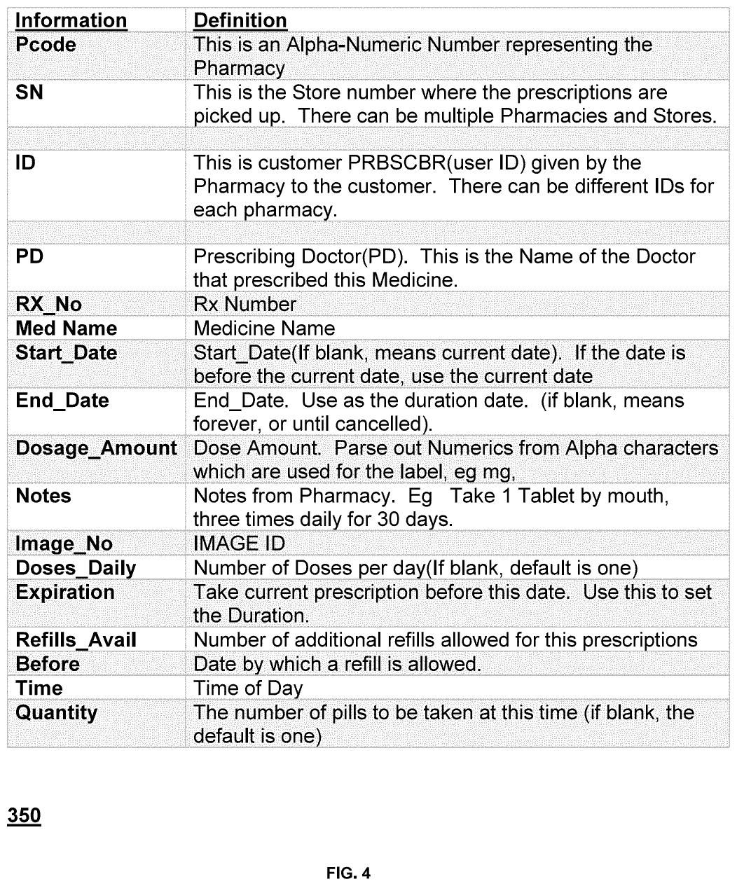

FIG. 4 shows an exemplary list 350 of data records downloadable from the pharmacy database during the procedure outlined in FIGS. 1 and 2. The exact list of data records and their format is determined in advance of allowing any users access to pharmacy databases. This ensures that all records are in a standard form so that no normalization or conversion step is required between the pharmacy records and the application. This further ensures there will be no errors in translation between the two systems and provides a uniform experience with all data from all pharmacies.

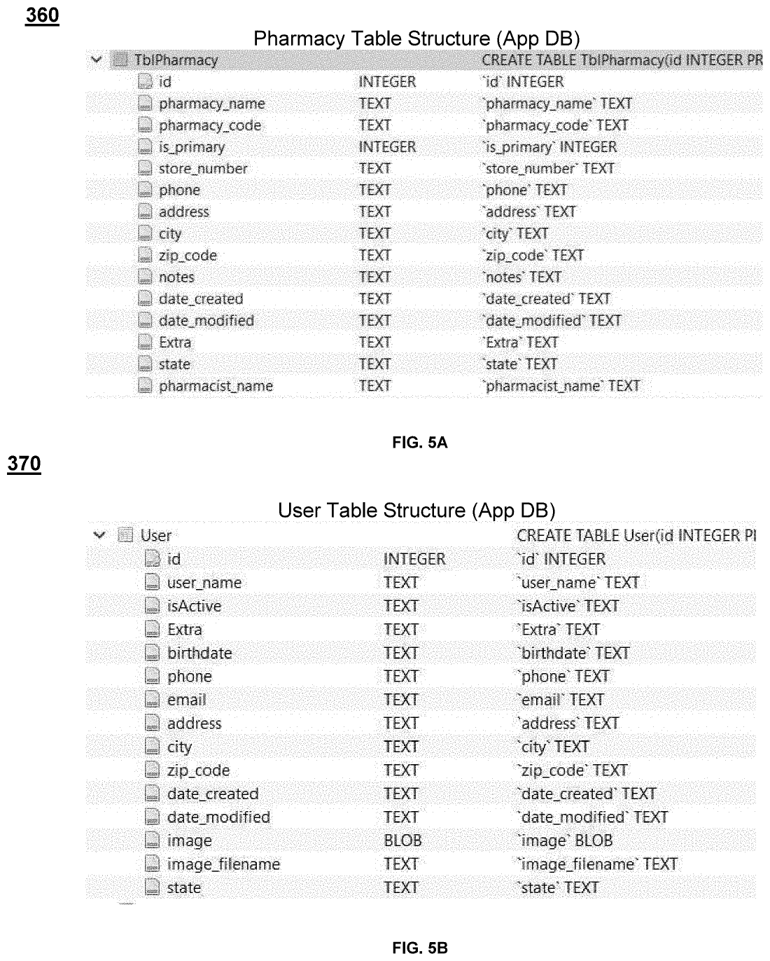

FIG. 5 shows, but is not restricted or limited to, two tables 360, 370 listing exemplary definitions existing in the IPP application. By standardizing which data is expected in which formats before attempting to download the same from the pharmacies, the experience for the user is cleaner, more reliable and free of errors that are introduced in the data scraping process of the previous systems (prior art) and methods.

The embodiments of the present invention are much simpler than previous systems (prior art) as shown in FIGS. 1 and 2 and provide several advantages. First, the instant system can download information for a user without forcing the user to sign up at that particular pharmacy and then manually enter the information into the application. Second, with the embodiments of the present invention, the pharmacy can provide additional information that might not be available to a user on the pharmacy platform. For example, the pharmacy might provide a photo and name of the pharmacist, or more information regarding interactions, warning messages, and/or additional information that may not normally be provided in the pharmacy platform. Third, it is also easier to implement the instant method within the application because the received data 109 is in the format the application expects. To the contrary, with the previous systems (prior art) and methods, if an application logs into the pharmacy it must create the normalization method for the data as it is imported. If the data or webpage changes form or display order or the like, the application fails to work properly. Fourth, the instant method is more reliable such that the probability of obtaining, storing and displaying correct data from the pharmacy is greatly enhanced by using the present file format to exchange information in comparison to the previous systems (prior art) and methods which attempt to match and read data intended for a browser as opposed to an application.

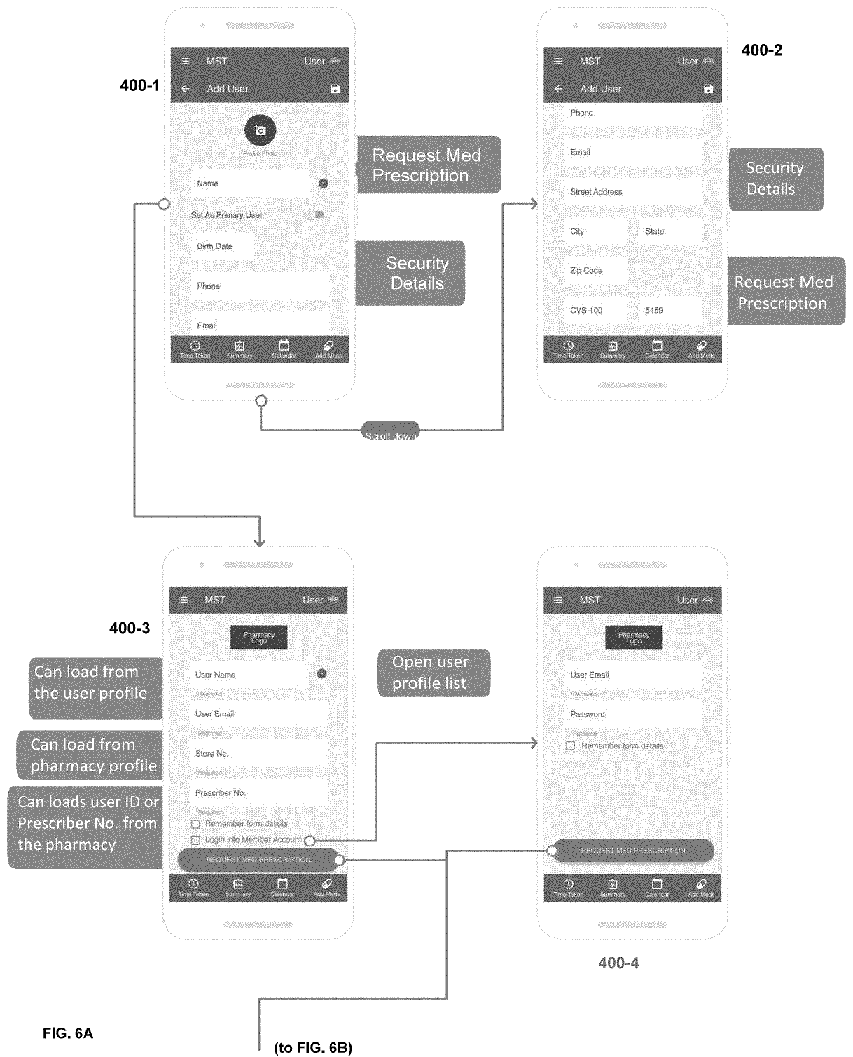

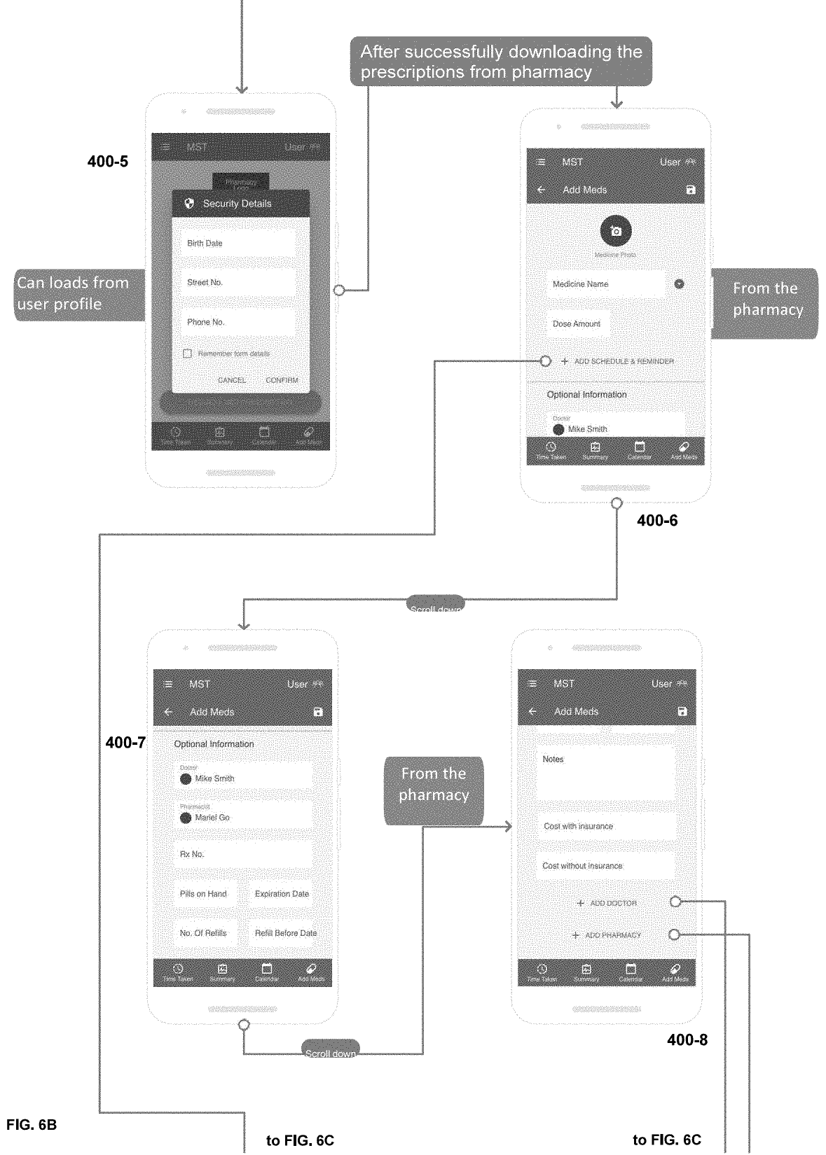

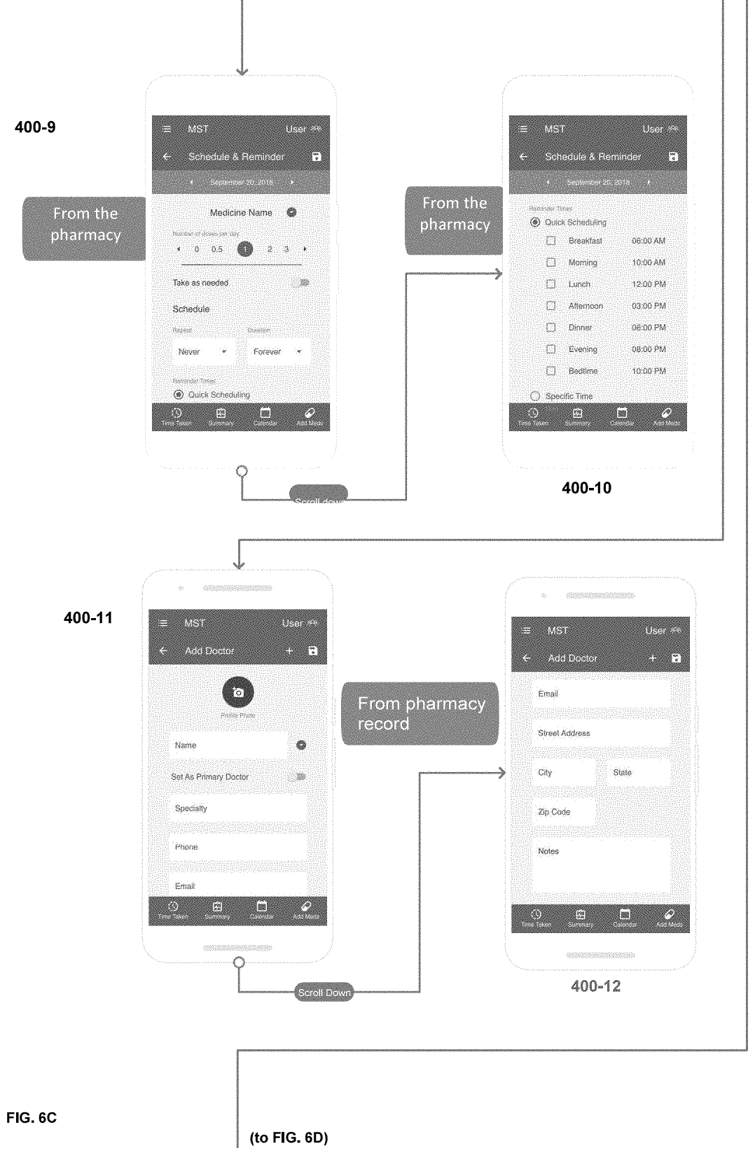

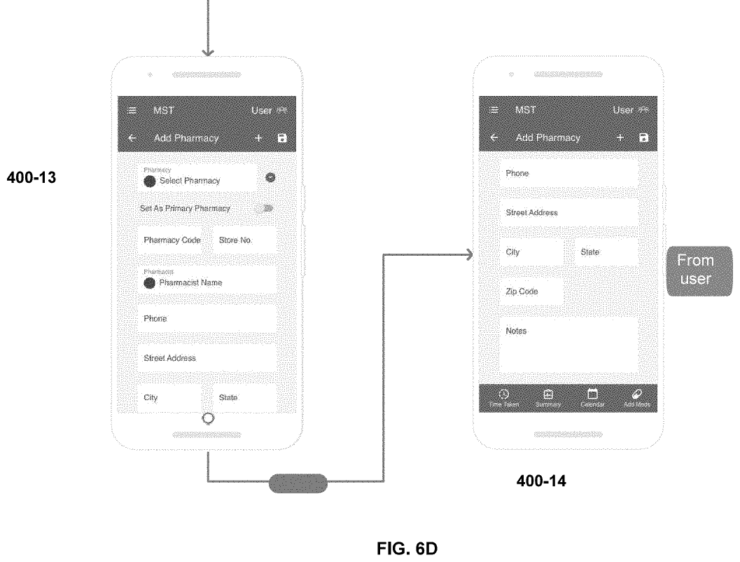

FIGS. 6A through 6D, as described in more detail below, show screen shots detailing functionality and user interaction. FIG. 6A shows a series of screen shots 400-1 through 400-4 of data entry pages for users to enter information. The user enters personal identifying data including name, birthdate, phone number, street address and email address. This personal information is securely stored in the user profile, along with a security number provided by the application that identifies the pharmacy being added for the user. The security number may include symbols, shapes, numbers, letters, words, designs, etc. There can be more than one individual profile stored in one instance of the application. Upon saving this data, the application prompts the user to enter a password for that user profile as identified by the email address. The information shown in FIG. 6A need not be entered every time the user accesses their profile. Screen shots 400-3 and 400-4 show screen shots after the individual has saved the personal profile information shown in screen shots 400-1 and 400-2. Screen shot 400-3 shows how an individual can select a profile from a list in the top input area. If the user does so, they are forced to enter a password to access the data as shown on screen shot 400-4. The selected profile then causes the remaining fields to be automatically filled. These are the login details saved earlier using the functionality of screen shots 400-1 and 400-2, the user has logged into their local profile and he or she may download prescription data.

If the user does not enter a local profile, they need to enter the data into the form shown in screen shot 400-5 of FIG. 6B. Upon confirmation of these details, the process moves along the arrow to the “Add Meds” screen shots 400-6 through 400-8. Screen shots 400-6 through 400-8 allow the user to add the downloaded medicines into the scheduling application. The information fields for the prescription are automatically filled from the data previously downloaded from the pharmacy. The user may have the chance to override the auto-filled information, or to take several other actions. Screen shots 400-9 and 400-10 show reminders scheduled. The “Add Doctor” link of screen shot 400-8 opens access to screen shots 400-11 and 400-12 of FIG. 6C. Here, the prescribing doctor's name is auto-filled from the prescription information, and the user can add other information if desired. The “Add Pharmacy” link of screen shot 400-8 takes the user to screen shots 400-13 and 400-14 shown in FIG. 6D. Here, the pharmacy code is set automatically by the application (since the application has the code from the pharmacy from which the data has been downloaded), and the store number and the pharmacist's name are data from the download performed previously. The user's information populates the rest of the fields from the profile entered into the application previously.

The “Add Schedule & Reminder” link of screen shot 400-6 takes the user to screen shots 400-9 and 400-10 of FIG. 6C where all the dosage and frequency directions from the pharmacy data are auto-filled. The user again has the opportunity to either accept the scheduled reminder with the default settings or override the auto filled data with custom entries.

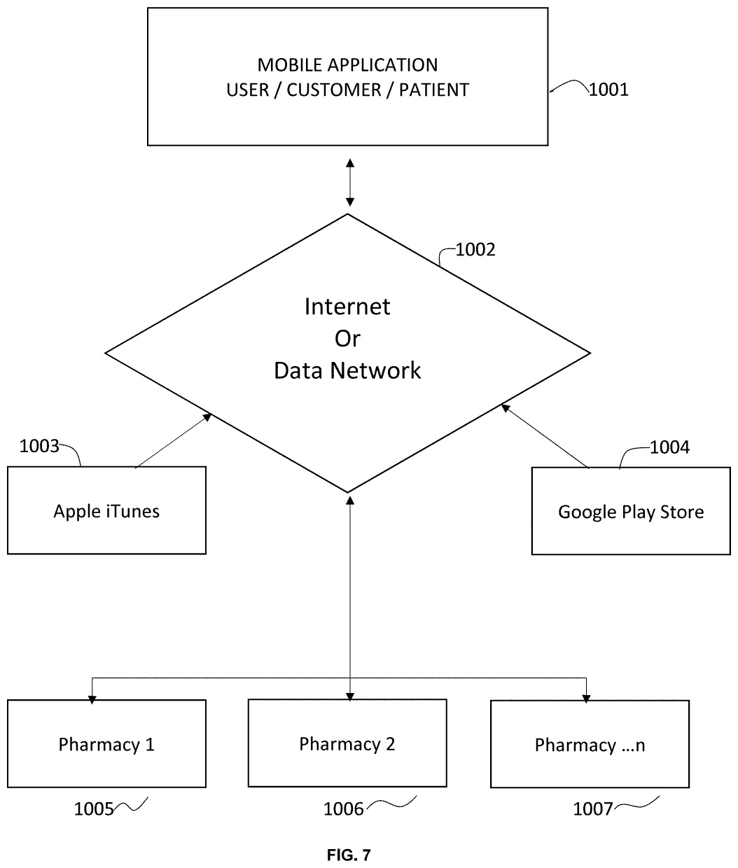

FIG. 7 shows a chart detailing the flow of a new user connecting to the Internet, downloading the mobile application to connect with pharmacy data for import into the mobile application according to the embodiments of the present invention. A user initially downloads the application with IPP implemented. The user may use a mobile device 1001 with an Internet or data network connection 1002 to connect to iTunes 1003 or the Google Play Store 1004 where the mobile application which facilitates the embodiments of the present invention can be downloaded. The application downloads a user's prescription automatically by communicating with the pharmacy database server in a pre-defined standard (mutually agreed upon) format. The embodiments of the present invention define the standard for communicating between the application and the pharmacy where each pharmacy is assigned a security code and/or process. The pharmacy security code and/or process is used to ensure communications are between authorized parties to exchange prescription information.

Prior to communicating from the application downloaded on the user's mobile device 1001, an associated security code and/or process is assigned by the application to communicate with the pharmacy's database server. If the mobile application lists or communicates with multiple pharmacies, the user must first select from a list of pre-approved pharmacies with an associated security code and/or process. After selecting a pre-approved pharmacy that matches the user's pharmacy, the user must enter some basic information to identify themselves to the pharmacy's database server. This information can be setup in a user profile page and pharmacy profile page of the application. Once set up in these pages, the application can auto-fill or the user can manually enter the requested data into a designated application IPP form. The requested IPP entry data is saved so that the IPP form is pre-populated the next time the user needs to request additional or updated information communicated between the application and pharmacy's database server saving the user time spent entering data into the IPP form. The user may then request (e.g., select a button) to send this info to the participating pharmacies 1005, 1006, 1007. Upon receipt of the information, the select pharmacy's database server verifies the user is a customer and has prescription records. Once the pharmacy database server verifies the request is from a pharmacy customer, the pharmacy's database server returns an acknowledgement to the application 1001.

The application 1001 then displays a security details form. Based on preference of the pharmacy, the application user either enters his or her membership login and password, birthdate, first 4 digits of their street address and telephone number, and/or any other security details agreed to between the application and the pharmacy. The user then requests (e.g., selects a button) to send this info to the pharmacy's database server. The information in the form can be auto-filled from the user profile form in the application or can be saved and auto-filled from prior requests to the pharmacy. Once the pharmacy database server verifies the security details, the pharmacy transmits the prescription information in a predefined file format that has been harmonized between the pharmacy's database server and the application. The application imports the information received from the pharmacy database server and populates portions of the application database. The prescription information entered into the application database can then be used to add the user's prescription medicines to the application's schedule of reminders along with other information about the prescription and/or pharmacy (e.g., medicine name, Rx number, medicine photos, scheduled reminders, expiration dates, name of pharmacy pharmacist, prescribing doctor, medicine warnings, etc.). The amount of information provided by the pharmacy's database server can be customized by the pharmacy to include any information the pharmacy normally provides on prescription bottle labels, or as the pharmacy deems appropriate.

Although the invention has been described in detail with reference to several embodiments, additional variations and modifications exist within the scope and spirit of the invention as described and defined in the following claims.

We claim:

1. A computer-implemented system comprising:
memory and one or more hardware processors;
a software application running on a mobile smart device;
a communication link between said mobile smart device and one or more pharmacy database servers, said one or more pharmacy database servers maintaining user-related prescription data or pharmacy information;
a user interface generated by said software application on said mobile smart device;
wherein said software application and pharmacy database servers cooperate to:
verify identity of a user and access to pharmacy database servers by said software application, said verification requires said user to be a customer of said pharmacy but does not require said user to register a user account with a pharmacy's web or mobile interface; and
transmit said user-related prescription data to said mobile smart device, said user-related prescription data formatted in a pre-established manner compatible with said software application.

2. The computer-implemented system of claim 1 wherein said transmitted user-related prescription data is stored in a database associated with said software application.

3. The computer-implemented system of claim 2 wherein said stored user-related prescription data is used to create scheduling reminders for said user on said mobile smart device.

4. The computer-implemented system of claim 1 wherein a security code is used to verify said access to said pharmacy database servers by said software application.

5. The computer-implemented system of claim 1 wherein said user-related prescription data is customized by said pharmacy.

6. The computer-implemented system of claim 1 wherein said user-related prescription data or pharmacy information includes at least one of pharmacist name, pharmacist photo and/or pharmacy promotions.

7. A computer-implemented method comprising:
running a software application running on a mobile smart device, said mobile smart device having memory and one or more hardware processors;
generating a user interface on said mobile smart device;
establishing a communication link between said mobile smart device and one or more pharmacy database servers, said pharmacy database servers maintaining user-related prescription data;
formatting said user-related prescription data in a pre-established manner so that said user-related prescription data is compatible with said software application;
verifying identity of a user and access to pharmacy database servers by said software application, said verification requires said user to be a customer of said pharmacy but does not require said user to register a user account with said pharmacy's web or mobile interface; and
transmitting said user-related prescription data to said mobile smart device.

8. The computer-implemented method of claim 7 further comprising storing said transmitted user-related prescription data in a database associated with said software application.

9. The computer-implemented system of claim 7 further comprising creating scheduling reminders for said user on said mobile smart device.

10. The computer-implemented method of claim 7 further comprising using a security code to verify said access to said pharmacy database servers by said software application.

11. The computer-implemented method of claim 7 wherein said user-related prescription data is customized by said pharmacy.

12. The computer-implemented method of claim 7 wherein said user-related prescription data or pharmacy information includes at least one of pharmacist name, pharmacist photo and/or pharmacy promotions.

13. A tangible non-transitory machine readable medium storing instructions that, when executed, cause a computing device to perform a method, the method comprising:
generating a user interface on a mobile smart device;
establishing a communication link between said mobile smart device and one or more pharmacy database servers, said pharmacy database servers maintaining user-related prescription data;
formatting said user-related prescription data in a pre-established manner so that said user-related prescription data is compatible with a software application running on said mobile smart device;
verifying identity of a user and access to pharmacy database servers by said software application, said verification requires said user to be a customer of the pharmacy but does not require said user to register a user account with a pharmacy's web or mobile interface; and
transmitting said user-related prescription data to said mobile smart device.

14. The tangible non-transitory machine readable medium of claim 13 further comprising instructions that, when executed, cause said computing device to perform a method, the method further comprising storing said transmitted user-related prescription data in a database associated with said software application.

15. The tangible non-transitory machine readable medium of claim 13 further comprising instructions that, when executed, cause said computing device to perform a method, the method further comprising creating scheduling reminders for said user on said mobile smart device.

16. The tangible non-transitory machine readable medium of claim 13 further comprising instructions that, when executed, cause said computing device to perform a method, the method further comprising using a security code to verify said access to said pharmacy database servers by said software application.

17. The tangible non-transitory machine readable medium of claim 13 further comprising instructions that, when executed, cause said computing device to perform a method, the method further comprising allowing said pharmacy to customize said user-related prescription data.

18. The tangible non-transitory machine readable medium of claim 13 wherein said user-related prescription data or pharmacy information includes at least one of pharmacist name, pharmacist photo and/or pharmacy promotions.
